(19) United States
(12) Patent Application Publication
(10) Pub. No.: US 2007/0065418 A1
(43) Pub. Date: Mar. 22, 2007

(54) Method and device for cellular therapy

(76) Inventors: Franco Vallana, Torino (IT); Giovanni Rolando, Chivasso (Torino) (IT); Maria Curcio, Saluggia (Vercelli) (IT); Andrea Grignani, Chieri (Torino) (IT)

Correspondence Address: POPOVICH, WILES & O'CONNELL, PA;650 THIRD AVENUE SOUTH, SUITE 600, MINNEAPOLIS, MN, 55402, US

(21) Appl. No.: 11/524,087

(22) Filed: Sep. 20, 2006

(30) Foreign Application Priority Data
Sep. 20, 2005 (IT) TO2005A000650
Mar. 17, 2006 (EP) 06111309.8

(51) Int. Cl. A61M 31/00; A61M 37/00; A61K 35/12

(52) U.S. Cl. 424/93.7; 604/95.03

(57) ABSTRACT

A device and method for performing a therapeutic treatment at a treatment site in a patient's vessel, the device configured to be introduced by means of a catheter, the device including an expandable portion capable of being expanded between a contracted position and a deployed position at the treatment site, such as to achieve an angioplasty treatment at the site, and a conveyor portion capable of receiving cells for cellular therapy and delivering the cells to the treatment site such that the angioplasty and cellular therapy procedures are performed substantially simultaneously.

The applications from which this application claims foreign priority, Italian Patent Application No. TO2005A000650, filed Sep. 20, 2005, and European Patent Application No. 06111309.8, filed Mar. 17, 2006, are hereby incorporated by reference.

### FIELD OF THE INVENTION

The present invention relates generally to techniques for cellular therapy and, in particular to the application of cellular therapy to treating the cardiac muscle or cardiac vessels.

### BACKGROUND OF THE INVENTION

Techniques which include cellular therapy usually entail the utilisation of cells that, distributed at a target site such as a cardiac site, are able to “repair” the site itself, for example repairing parts of the damage suffered by the cardiac muscle due to an ischemic event (infarction). The cells in general are different cells from those that constitute the cardiac muscle. In most cases they are autologous cells, that is from the patient him/herself, such as stem cells.

Although these are rather recent techniques, there is considerable literature relating to such therapy, as shown by the following documents:
P. Menasché et al., “Myoblast transplantation for heart failure”, Lancet 357:279-280 (Jan. 27, 2001);
P. Menasché et al., “Scientists Inject Arm Muscle Cells into Damaged Heart”, Associated Press, 30 May 2001;
P. Menasché et al., “First Percutaneous Endovascular Case of Heart Muscle Regeneration Completed with Bioheart's MyoCell™ Product”, PRNewswire, 30 May 2001;
R. M. El Oakley et al., “Myocyte transplantation for cardiac repair: A few good cells can mend a broken heart”, Annals of Thoracic Surgery, 71:1724-1733 (2001);
K. A. Jackson et al.: “Regeneration of ischemic cardiac muscle and vascular endothelium by adult stem cells”, Journal of Clinical Investigation, 107(11): 1395-1402 (June 2001);
N. N. Malouf et al., “Adult-derived stem cells from the liver become myocytes in the heart in vivo”, American Journal of Pathology, 158(6): 1929-1935 (June 2001);
D. Orlic et al., “Bone marrow cells regenerate infarcted myocardium”, Nature, 410:701-705 (Apr. 5, 2001);
A. A. Kocher et al., “Neovascularization of ischemic myocardium by human bone-marrow-derived angioblasts prevents cardiomyocyte apoptosis, reduces remodeling and improves cardiac function”, Nature Medicine, 7(4):430-436 (April 2001);
J. S. Wang et al., “Marrow stromal cells for cellular cardiomyoplasty: feasibility and potential clinical advantages”, The Journal of Thoracic and Cardiovascular Surgery, 120(5):999-1006 (November 2000);
M. Scorsin et al., “Comparison of the effects of fetal cardiomyocyte and skeletal myoblast transplantation on postinfarction left ventricular function”, The Journal of Thoracic and Cardiovascular Surgery, 119(6): 1169-1175 (June 2000); and
T. Siminiak et al., “Myocardial Replacement Therapy”, Circulation, 108(10):1167-1171 (Sep. 9, 2003).

In general, dissemination of cells in cardiac cellular therapy comes about by injecting the cells into the cardiac muscle, for example during heart surgery, or in a cardiological environment, placing the cells by use of an intra-coronary catheter.

Although such treatment is undoubtedly promising, the results achieved to date have not been entirely satisfactory. Tests have shown, for example, that there is a risk of generating arrhythmia or of achieving results that are of poor quality. In particular, for example, one problem is that the final destination of the cells used for therapy is not entirely clear nor under control. More specifically, when stem cells are used some researchers believe that the stem cells themselves might undesirably reach sites other than the treatment site, and stimulate aberrant or in any case undesirable cell growth.

At least during these early years, the attention of researchers and experimenters appears to have been concentrated primarily on the characteristics of the cellular material utilised for therapy. A significant line of research has been aimed at identifying which cells are preferable, as a function of the type of therapy. At present, the prevailing tendency is to prefer stem cells of skeletal origin harvested, for example, from bone marrow rather than muscular cells. Another line of investigation is linked to possible treatments that the cells undergo after harvesting in view of their intended use. For example, the cells may be harvested and caused to grow in vitro in view of their use for therapy, or alternatively implanted immediately after harvesting.

Another extensive line of research is linked to the possibility of associating chemical substances such as, for example, cytokines to the cells used for treatment. It is thought that cytokines are able to stimulate the stem cells present in the heart.

One of the major problems in achieving satisfactory results is connected with the survival of cells in the treated tissue. The possibility of enhancing cell survival offered by associating drugs or growth factors to the cells are interesting from this standpoint. This is also true with regard to the possible addition to the cells of bio-materials able to ensure survival of the implanted cells.

To date, less attention has been paid to the specific modalities of implantation of the cells used in the therapy. Several delivery possibilities are known. The delivery of the cells through the distal portion of a catheter introduced into a patient's body through the peripheral vascular system (for example through the femoral artery), is an essentially non-invasive method which appears to be preferred over direct injection into the heart performed during heart surgery.

### SUMMARY OF THE INVENTION

An object of the present invention is to provide an improved device for the performance of cellular therapy such as, for example, cardiac cellular therapy. According to the present invention this object is achieved by providing and using a device having the characteristics set forth specifically in the attached claims which form an integral part of the disclosure of the invention provided herein.

One preferred embodiment of the invention is a device for cellular therapy suitable for introduction through a catheter towards a treatment site (V). The device comprises an expandable portion, capable of being expanded between a contracted position and a deployed position at the treatment site such that the device is capable of being used to perform an angioplasty treatment at said site. The device further comprises a conveyor portion, capable of receiving cells for cellular therapy and of delivering the cells at the treatment site concurrently with the performance of the angioplasty operation. In one embodiment the conveyor portion is coupled to a supply line for supplying cells for cellular therapy towards said conveyor portion, and/or contains a charge comprising cells for cellular therapy.

The device according to the present invention thus makes it possible to perform, simultaneously, during a non-invasive intervention (that is through a catheter) two concomitant actions on a treatment site (typically a blood vessel), namely (1) implantation at the treatment site of the cells destined to achieve the therapy, and (2) an angioplasty treatment, with an effect antagonistic to the stenosis of the treated vessel.

Without intending to restrict this application to any specific theory in this connection, the applicant has reason to believe that the quality of the results achievable with the device and methods according to the present invention is linked to the fact that the device and methods utilize both angioplasty and cell therapy simultaneously. In other words the device and methods of the present invention achieve simultaneous revascularisation through, for example, angioplasty and tissue regeneration through cell therapy stimulus.

In one preferred embodiment of the invention the action of implanting the cells at the treated site is achieved in a selective fashion such that virtually all the cells used for treatment purposes are targeted towards the treated tissue and not dispersed in the blood stream. In this way the risk that these dispersed cells may be lost for the purposes of achieving treatment at the desired location, or are misdirected to other locations of the patient's body where they are capable of inducing aberrant growth is reduced.

Again, the applicants have reason to believe that the concomitant performance of an angioplasty operation and of the implantation of cells with therapeutic effect means that the treated tissue receives the cells in a condition in which the tissue itself (typically a blood vessel, such as a coronary vessel) is maintained in a condition of at least slight expansion. During such a procedure the vessel wall is at least partially extended with regard to its normal physiological dimensions. Although it is not at present possible to demonstrate this fact in absolute terms, it may be hypothesised that the greater efficacy of the treatment according to the present invention is linked to the fact that, in the extended or slightly expanded condition, the vessel wall is more easily permeable with regard to the implanted cells.

In one embodiment the invention is a device for performing a therapeutic treatment at a treatment site in a patient's vessel. The device comprises a catheter, means associated with a distal portion of the catheter for performing an angioplasty treatment at the treatment site, and means associated with the distal portion of the catheter for performing cellular therapy at the treatment site substantially simultaneously with the angioplasty treatment. The means for performing an angioplasty treatment may comprise at least one expandable balloon, two or more expandable balloons, and in some embodiments comprises three expandable balloons. The means for performing an angioplasty treatment comprises a stent which may be balloon expandable or self-expanding. The device may further comprise a source of cells and the means for performing cellular therapy may comprise a tubular member which defines a path from the source of cells to the treatment site. The tubular member may comprise a lumen within the catheter. The catheter may have at least one opening from the lumen in the distal portion through which cells from the source of cells can be administered to the treatment site during the cellular therapy. In some embodiments the stent comprises a radially external surface provided with a plurality of reservoirs which contain cells used in performing the cellular therapy.

In another embodiment the invention is a device for performing a therapeutic treatment at a treatment site in a patient's vessel. The device comprises a catheter having proximal and distal ends, a distal portion, an intermediate portion, a lumen, and at least one expandable balloon associated with the distal portion, the at least one expandable balloon configured for performing an angioplasty procedure at the treatment site. The catheter further has at least one opening in the distal portion between the lumen and an exterior of the catheter and a source of cells for use in performing cellular therapy at the treatment site connected at a proximal end of the lumen such that the cells are delivered to the treatment site while the angioplasty procedure is being performed. The at least one expandable balloon may comprise two or more expandable balloons, and in some embodiments comprises three expandable balloons. The device may further comprising a stent mounted on the at least one expandable balloon. The stent may be balloon expandable or self-expanding.

In a further embodiment the invention is a device for performing a therapeutic treatment at a treatment site in a patient's vessel comprising a catheter having proximal and distal ends, a distal portion, and an expandable stent associated with the distal portion, the expandable stent including a surface having a plurality of reservoirs containing cells, the stent being configured upon expansion for simultaneously performing an angioplasty procedure and cell therapy at the treatment site. The stent may be balloon expandable or self-expanding. The device may further comprise a source of cells and the catheter may include a lumen which defines a path from the source of cells to the treatment site. The catheter may be provided with at least one opening from the lumen in the distal portion through which cells from the source of cells can be administered to the treatment site during the cellular therapy. In one embodiment the catheter has at least two balloons and the openings are positioned longitudinally on the catheter between the at least two balloons such that the cells are delivered during cellular therapy into a space enclosed by the catheter, the expanded at least two balloons and a wall of the vessel.

In a still further embodiment the invention is a method for performing a therapeutic treatment at a treatment site in a patient's vessel. The method comprises providing a catheter having proximal and distal ends, a distal portion, an intermediate portion, a lumen, and at least one expandable balloon associated with the distal portion, the at least one expandable balloon configured for performing an angioplasty procedure at the treatment site, the catheter having at least one opening in the distal portion between the lumen and an exterior of the catheter. The method further includes providing a source of cells for use in performing cellular therapy at the treatment site and connecting the source of cells to a proximal end of the lumen of the catheter. The catheter is advanced through the patient's vessel until the at least one expandable balloon is at the treatment site. The at least one balloon is expanded at the treatment site while simultaneously delivering cells to the treatment site to thereby simultaneously perform an angioplasty procedure and cell therapy at the treatment site. In this embodiment the at least one expandable balloon may comprises two or more expandable balloons, or may comprise three expandable balloons. A stent may be mounted on the at least one expandable balloon. The stent may be balloon expandable or self-expanding and may be provided with a surface having a plurality of reservoirs on a radially external surface. The reservoirs contain cells used in performing the cellular therapy.

### BRIEF DESCRIPTION OF THE DRAWING

The invention will now be described, as a simple example without limiting intent, with reference to the attached drawings, in which:

FIG. 1 illustrates a first embodiment of the device of the invention.

FIG. 2 illustrates a possible varied embodiment of the device of FIG. 1.

FIG. 3 illustrates the device of FIG. 1 in conditions of use.

FIG. 4 is a section along the line IV-IV of FIG. 3.

FIG. 5 illustrates another embodiment of the device according to the present invention.

FIG. 6 is a section along the line VI-VI of FIG. 5.

### BRIEF DESCRIPTION OF THE PREFERRED EMBODIMENTS

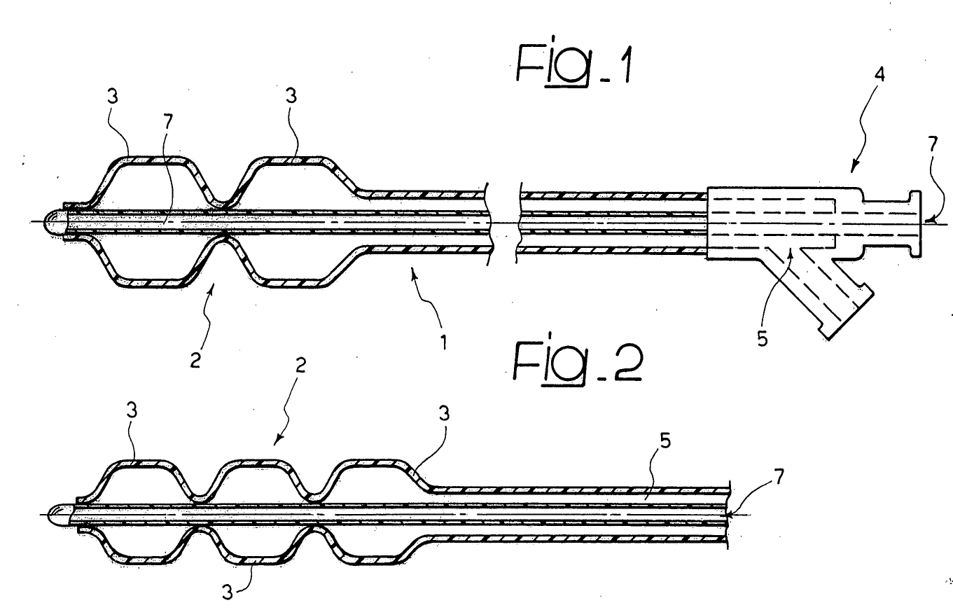

The device 1 illustrated in FIGS. 1 to 4, is configured as a catheter. In particular, as described herein the catheter is a “balloon” catheter. Since the construction of such catheters are known the details of construction of the catheter will not be specified except as necessary for an understanding of the invention.

Catheter 1 has at its distal end 2, one or more “balloon” elements 3 that can be inflated by means of a fluid made to flow from the proximal end 4 (or, in alternative, from an intermediate portion) of the catheter 1 through a tubular cavity or lumen 5. The tubular cavity 5 extends from the proximal end to the balloon of the catheter or, in the embodiment in which more than one balloon 3 is present (for example two or three balloons, as in the devices illustrated in FIGS. 1 and 2) it extends to the balloon situated more distant from the distal end of the catheter. In other words the balloon that is closest to the proximal end of the catheter. In this latter case, the various balloons are in communication (as can better be understood in the sectional view shown in FIG. 4) through apertures 6 for the passage of the inflation fluid. The balloon or balloons 3 present extremities that are narrow, where the balloon or balloons 3 are in close proximity with a central tube 7.

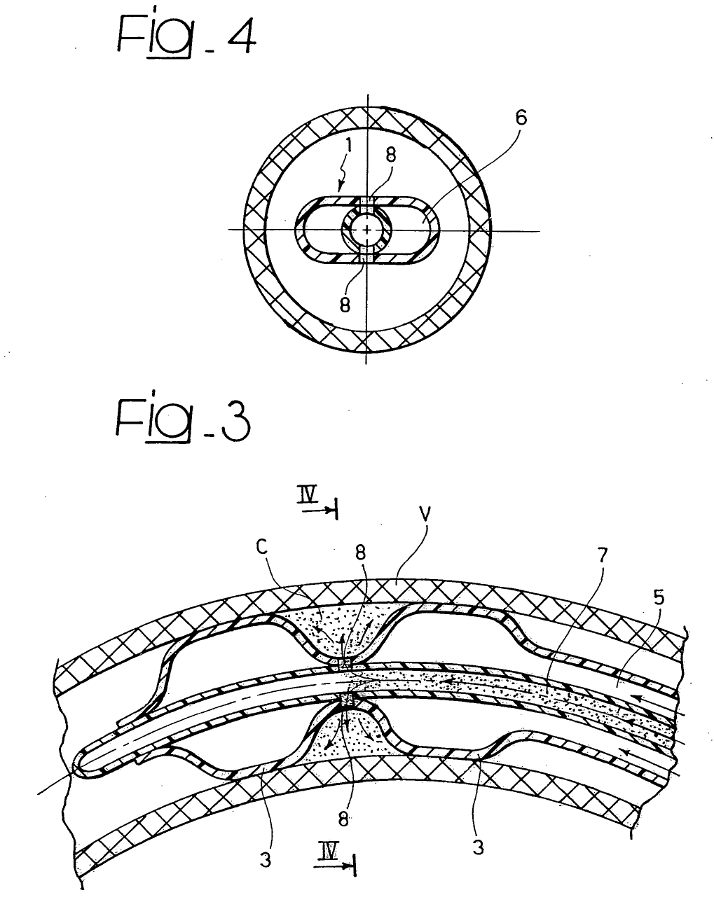

The central tube 7 extends distally from the proximal end 4 (or from an intermediate portion) of the catheter. Tube 7 has one or more openings 8 (not visible in FIGS. 1 and 2 but clearly visible in FIGS. 3 and 4) communicating with the outside of the catheter 1 at locations corresponding with the zone where the balloon or the balloons 3 are in close proximity with tube 7. The opening or openings 8 to diffuse the cells C towards the treatment site is/are situated in an adjacent position to the balloon element or elements 3. If only one balloon 3 is present, a preferred embodiment entails the presence of two openings or two groups of openings 8 situated at the two extremities (distal and proximal) of the balloon 3.

If the catheter 1 has a plurality of balloons (for example two or three, as illustrated in FIGS. 1 and 2) the openings 8 are located in intermediate positions between adjacent balloons 3. Preferably, openings 8 are positioned only in such intermediate positions. In other words, the openings 8 open onto the outside of the catheter 1 at a location (in the sense of the longitudinal development of the catheter 1) between adjacent balloons 3.

The materials that can be used to make the central tube 7 and the balloons 3 are those commonly used in catheter technology. Since these devices are used as disposable medical devices materials approved for medical and surgical use such as certain plastic materials are preferred.

The method of use of the device illustrated in FIGS. 1 to 4 includes introducing the catheter into the patient's body in the usual way, for example through the femoral artery, and guiding the catheter through the vessel until a distal portion 2 reaches the site to be treated (for example a stenotic coronary vessel V that has caused an ischaemic episode). The catheter is introduced with the balloons in a radially retracted condition (that is with the balloons 3 “deflated”, in general wrapped around the extremity of the catheter). Once the treatment site has been reached, the inflation fluid is introduced into the tubular cavity 5 under pressure, such that the balloons 3 inflate and expand radially, in the condition illustrated in FIGS. 1 to 4. In particular, FIG. 3 shows the angioplastic action performed on the treated vessel, indicated as V, due to radial expansion of the balloons 3.

At the same time as the angioplasty treatment is being performed, cells C for cellular therapy (such as for example stem cells for cardiac cellular therapy) are introduced at or adjacent the treatment site. The cells are introduced using known means, for example with a syringe or similar instrument, into the central tube 7 and caused to travel distally until they reach the openings 8. From these openings, the cells diffuse outside the catheter 1 thus reaching the walls of the blood vessel V where they perform their therapeutic function. Again, this cellular therapy is advantageously performed substantially simultaneously with the angioplasty treatment derived from dilation of the balloons 3.

The fact that the opening or openings 8 are situated in an adjacent position to the balloon or balloons 3 and that they open to the outside of the catheter 1 in a zone situated longitudinally between adjacent balloons 3 means that the cells C are confined in a chamber created between adjacent balloons that, expanding radially, are in contact with the wall of the vessel V, and the wall of the vessel itself, thus avoiding any undesired dispersion.

Obviously, the above-described actions must be performed in a manner that avoids creating excess pressure capable of having negative effects on the site treated. For example, after a first radial expansion of a device such as that illustrated in FIGS. 1 and 2, the balloons 3 are at least slightly deflated to allow some flow of blood from the chamber or chambers created between adjacent balloons into which the cells C have been introduced through the openings 8. The balloons 3 may then again be expanded radially. Alternatively, blood flow may be made possible by providing grooves (not shown) on the external surface of the balloons 3, extending for preference in a longitudinal direction with regard to the balloons themselves, that is in the general axial direction of the device 1.

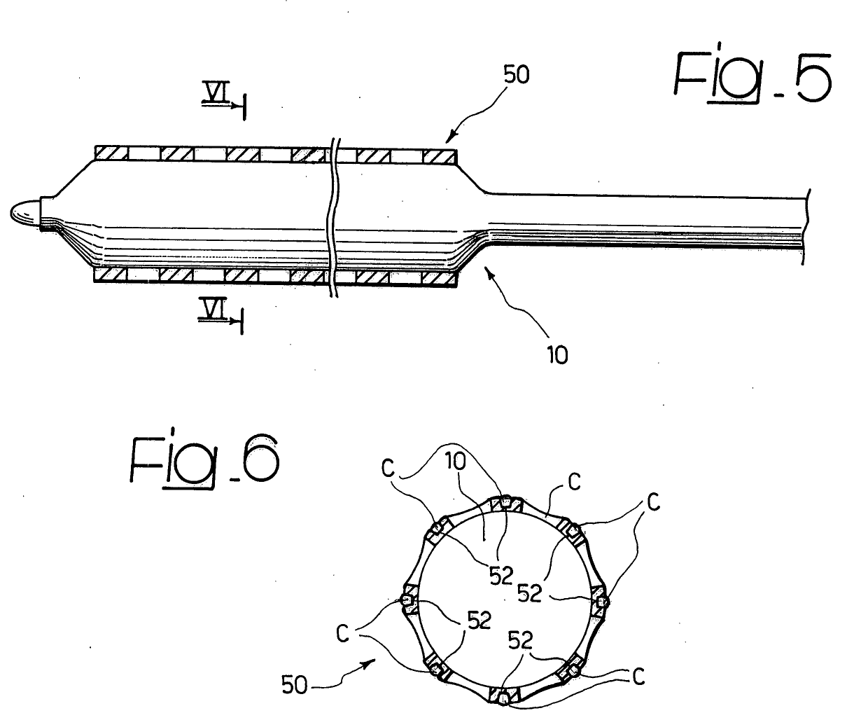

In the embodiment illustrated in FIGS. 5 and 6 the device 1 comprises a catheter 10 (of known type) to which is associated an angioplasty stent 50. The stent 50 may be, for example, of the type described in the documents EP-A-0 850 604, EP-A-1 181 903, EP-A-1 277 449, EP-A-1 310 242, and EP-A-1 449 546, all of which are assigned to the assignee of the present invention and all of which are incorporated herein by reference in their entirety. These stents are angioplasty stents capable of being mounted on a distal extremity of a balloon catheter and of then being positioned at the coronary site following the usual procedures for the use of angioplasty stents. The modalities of use and implantation of these devices are well-known to the technology and do not require detailed description herein.

In a further embodiment, not explicitly illustrated in the attached drawings, the stent 50 may be of the self-expanding type. This term in general indicates a stent made of super-elastic material (for example the material usually known as Nitinol), also designed to be introduced to the treatment site by means of a catheter. As is well-known in the technology, the difference between a stent such as that illustrated in FIG. 5 and a stent of the self-expanding type lies in the expansion mechanism during the implantation phase.

A stent such as that illustrated in FIG. 5 is designed to be mounted (“crimped” being the usual term) onto the deflated balloon of a balloon catheter. Once mounted on the catheter the stent is introduced into the patient's body and advanced to the treatment site. Having reached the treatment site, the balloon of the catheter is inflated thus expanding the stent 50 from the radially contracted condition in which it was crimped onto the balloon to a radially expanded position. Radial expansion of the stent causes firstly the dilation of the treated vessel, with consequent performance of an angioplasty operation. A second affect lies in the fact that, once expanded radially, the stent 50 conserves its radially-expanded condition (except for a slight phenomenon of radial contraction or recoil, induced by reaction against the wall of the treated vessel) thus maintaining the treated vessel in a condition of patency.

In the case of the stent 50 being of the self-expanding type, a distal portion of the introduction catheter is essentially comprised of a sheath or tubular member that initially covers the stent 50 maintaining it in a radially contracted condition. Once the site of implantation has been reached, the sheath is retracted in the distal to proximal direction to uncover and free the stent. By effect of its super-elastic characteristics (or “shape memory”) the stent assumes a radially expanded condition which corresponds—once again—to performing an action of angioplasty and maintaining the treated vessel in a condition of patency.

In both cases, the modalities for production and implantation of the various types of either balloon expandable or self expanding stents as described herein are well-known to technology and do not require a detailed description herein.

Whichever type of stent 50 is utilised, the significant characteristic with regard to the device 1 described herein is that the stent 50 possesses hollows 52 in the form of slots, notches, channels or grooves of various types appropriate to receive (if necessary together with other active principles, such as for example drugs contrasting re-stenosis) a charge of cells C for the performance of a cellular therapy, such as cardiac cellular therapy. Preferably the hollows 52 are included on the radially external surface of the stent, that is the surface which faces the wall of the treated vessel V. More preferably, the hollows 52 are included only on the radially external surface of the stent.

In contrast to the stents described in the last-quoted documents where the dimensions of the hollows or grooves in the surface are appropriate to receive active principles such as drugs, the hollows 52 of stents 50 must have dimensions compatible with the dimensions of the cells which are intended to be loaded therein. The hollows 52 essentially constitute reservoirs into which the cells C (and any other substances associated with them) are loaded before proceeding to the implantation of the stent.

Loading of the cells into the hollows 52 may come about in different ways. In some circumstances it is preferable that the cells C be loaded onto the stent 50 immediately before proceeding to implantation of the stent. For example, in one loading method the stent 50 is immersed in a mass of treatment cells C in such a fashion that said cells distribute themselves on the external surface of the stent and in particular inside the hollows 52 provided on the external surface of the stent 50. The immersion is done immediately before the stent is mounted onto the distal extremity of the catheter or, in the case of the catheter of an expandable balloon, also after having been crimped onto the distal extremity of the catheter.

In this fashion, only that fraction of cells C—many times less numerous—that deposit themselves on the parts of the outer surface of the stent surrounding the hollows or reservoirs 52 become dispersed along the penetration route of the catheter. The remainder of the cells (which are far more numerous) remain on the inside the hollows or reservoirs 52 and between the mesh, that is between the struts, of the stent 50. Consequently, when the stent 50 reaches the site of implantation and attains its radially expanded position, the cells C received into the hollows 52 are exposed to the wall of the treated blood vessel V as the sole administration route of the cells towards the treated cardiac site.

Additionally, the cells C that have been received within the openings between the mesh or struts of the stent 50 are in an approximately similar situation since, on the radially inner side of the stent, these openings are closed by the wall of the balloon of the catheter 10. Of note, since the intervention for positioning the stent usually involves expansion of the catheter 10 in several stages, the cells C have the possibility of being absorbed by the treated site before the blood flow is fully restored.

This manner of cell loading minimises (and virtually eliminates) the risk of an appreciable fraction of cells being washed away by the blood flow (restored by effect of the angioplasty action) and thus being wasted for treatment purposes. The high concentration of cells that can be achieved inside the hollows 52 means that the cells themselves may diffuse inside the vessel wall (and hence into the cardiac tissue) with marked penetration efficacy and thus marked treatment efficacy.

The cells C are in general applied/loaded onto the device 1 as “live” cells. In consequence, when, in the present description and in the attached claims, mention is made of applying and/or loading cells C onto the device 1 it is intended that such application and/or loading usually involves cells C associated with substances (for example, vehicles or matrices, cytokines, growth factors or other substances) destined to ensure the therapeutic efficacy of the cells C themselves.

Advantageously, the stent 50 is provided, in whole or in part, with a coating of biocompatible carbon-based material of the type described in documents such as, for example, U.S. Pat. No. 5,084,151, U.S. Pat. No. 5,133,845, U.S. Pat. No. 5,370,684, U.S. Pat. No. 5,387,247, or U.S. Pat. No. 5,423,886. The presence of such a coating inside the hollows 52 has been shown to be advantageous since it avoids any undesirable reaction between the cells C (and any substances associated with them) and the material of the stent, preserving the maximum therapeutic activity of the cells C. The presence of such coating on the inner surface of the stent 50 has been shown to be advantageous since it minimizes undesirable phenomena such as coagulation/thrombosing. The choice of providing the stent 50 with such a coating on its entire surface or only on the radially external surface or only on the radially internal surface is thus dictated by specific application requirements.

It should be understood that the embodiments disclosed herein represent presently preferred embodiments of the invention. Various modifications and additions may be made to these embodiments without departing from the spirit and scope of the invention as defined by the attached claims. In particular, it will be appreciated that the examples described and illustrated above correspond to situations in which the conveyor portion:

i) is coupled to a line (7) to supply cells (C) for cellular therapy towards said conveyor portion (FIGS. 1 to 4), or

ii) contains a charge comprising cells (C) for cellular treatment (FIGS. 5 and 6).

Nevertheless, the invention also includes embodiments in which such characteristics i) and ii) coexist, that is solutions in which the conveyor portion already contains a charge comprising cells for cellular therapy and at the same time is coupled to a line for supplying the cells for cellular therapy.

1. A device for performing a therapeutic treatment at a treatment site in a patient's vessel comprising:
a catheter;
means associated with a distal portion of the catheter for performing an angioplasty treatment at the treatment site; and
means associated with the distal portion of the catheter for performing cellular therapy at the treatment site substantially simultaneously with the angioplasty treatment.

2. The device of claim 1 wherein the means for performing an angioplasty treatment comprises at least one expandable balloon.

3. The device of claim 2 wherein the means for performing an angioplasty treatment comprises two or more expandable balloons.

4. The device of claim 3 wherein the means for performing an angioplasty treatment comprises three expandable balloons.

5. The device of claim 1 wherein the means for performing an angioplasty treatment comprises a stent.

6. The device of claim 5 wherein the stent is balloon expandable.

7. The device of claim 5 wherein the stent is self-expanding.

8. The device of claim 1 further comprises a source of cells and wherein the means for performing cellular therapy comprises a tubular member which defines a path from the source of cells to the treatment site.

9. The device of claim 8 wherein the tubular member comprises a lumen within the catheter.

10. The device of claim 9 wherein the catheter has at least one opening from the lumen in the distal portion through which cells from the source of cells can be administered to the treatment site during the cellular therapy.

11. The device of claim 5 wherein the stent comprises a surface provided with a plurality of reservoirs.

12. The device of claim 11 wherein the surface is a radially external surface.

13. The device of claim 11 wherein the reservoirs contain cells used in performing the cellular therapy.

14. A device for performing a therapeutic treatment at a treatment site in a patient's vessel comprising:
a catheter having proximal and distal ends, a distal portion, an intermediate portion, a lumen, and at least one expandable balloon associated with the distal portion, the at least one expandable balloon configured for performing an angioplasty procedure at the treatment site, the catheter having at least one opening in the distal portion between the lumen and an exterior of the catheter; and
a source of cells for use in performing cellular therapy at the treatment site connected at a proximal end of the lumen such that the cells are delivered to the treatment site while the angioplasty procedure is being performed.

15. The device of claim 14 wherein the at least one expandable balloon comprises two or more expandable balloons.

16. The device of claim 15 wherein the two or more expandable balloons comprise three expandable balloons.

17. The device of claim 14 further comprising a stent mounted on the at least one expandable balloon.

18. The device of claim 17 wherein the stent is balloon expandable.

19. The device of claim 17 wherein the stent is self-expanding.

20. The device of claim 17 wherein the stent comprises a surface provided with a plurality of reservoirs.

21. The device of claim 20 wherein the surface is a radially external surface.

22. The device of claim 21 wherein the reservoirs contain cells used in performing the cellular therapy.

23. A device for performing a therapeutic treatment at a treatment site in a patient's vessel comprising a catheter having proximal and distal ends, a distal portion, and an expandable stent associated with the distal portion, the expandable stent including a surface having a plurality of reservoirs containing cells, the stent being configured upon expansion for simultaneously performing an angioplasty procedure and cell therapy at the treatment site.

24. The device of claim 23 wherein the stent is balloon expandable.

25. The device of claim 23 wherein the stent is self-expanding.

26. The device of claim 23 further comprising a source of cells and wherein the catheter includes a lumen which defines a path from the source of cells to the treatment site.

27. The device of claim 26 wherein the catheter has at least one opening from the lumen in the distal portion through which cells from the source of cells can be administered to the treatment site during the cellular therapy.

28. The device of claim 27 wherein the catheter has at least two balloons and wherein the openings are positioned longitudinally on the catheter between the at least two balloons such that the cells are delivered during cellular therapy into a space enclosed by the catheter, the expanded at least two balloons and a wall of the vessel.

29. A method for performing a therapeutic treatment at a treatment site in a patient's vessel comprising:
providing a catheter having proximal and distal ends, a distal portion, an intermediate portion, a lumen, and at least one expandable balloon associated with the distal portion, the at least one expandable balloon configured for performing an angioplasty procedure at the treatment site, the catheter having at least one opening in the distal portion between the lumen and an exterior of the catheter;
providing a source of cells for use in performing cellular therapy at the treatment site;
connecting the source of cells to a proximal end of the lumen;
advancing the catheter through the patient's vessel until the at least one expandable balloon is at the treatment site;
expanding the at least one balloon at the treatment site; and
delivering cells to the treatment site during the expanding step to thereby simultaneously perform an angioplasty procedure and cell therapy at the treatment site.

30. The method of claim 29 wherein the at least one expandable balloon comprises two or more expandable balloons.

31. The method of claim 30 wherein the two or more expandable balloons comprise three expandable balloons.

32. The method of claim 29 further comprising a stent mounted on the at least one expandable balloon.

33. The method of claim 32 wherein the stent is balloon expandable.

34. The method of claim 32 wherein the stent is self-expanding.

35. The method of claim 32 wherein the stent comprises a surface provided with a plurality of reservoirs.

36. The method of claim 35 wherein the surface is a radially external surface.

37. The method of claim 35 wherein the reservoirs contain cells used in performing the cellular therapy.
